(19) United States
(12) Patent Application Publication
(10) Pub. No.: US 2020/0222475 A1
(43) Pub. Date: Jul. 16, 2020

(54) THE TRANSDERMAL USE OF CYANOBACTERIAL ALGAE, AND/OR EXTRACTS THEREOF, VIA LOW OR HIGH INTENSITY TRANSPORATION DEVICES FOR BEAUTY AND HEALTH ENHANCING PURPOSES

(71) Applicant: Stefano SCOGLIO, Urbino (IT)

(72) Inventors: Stefano SCOGLIO, Urbino (IT); Gabriel Dylan SCOGLIO, Urbino (IT)

(21) Appl. No.: 16/490,565

(22) PCT Filed: Sep. 27, 2018
(86) PCT No.: PCT/EP2018/073036

§ 371 Date: Sep. 2, 2019

Related U.S. Application Data
(60) Provisional application No. 62/551,768, filed on Aug. 29, 2017.

(51) Int. Cl. A61K 35/748; A61K 41/00; A61K 9/00; A61K 9/14; A61K 9/06; A61K 47/06; A61K 38/43

(52) U.S. Cl. CPC A61K 35/748; A61K 41/0047; A61K 9/0014; A61K 38/43; A61K 9/06; A61K 47/06; A61K 9/14

(57) ABSTRACT

The present invention refers to the transporation, or force-transdermal vehiculation, of cyanobacterial algae or extracts thereof, through electroporation, sonoporation (sound), photoporation (light), radioporation (radio frequency), and through any other transporation tool for enhancing the transdermal penetration of cyanobacterial molecules via the transmission of some force, be it electric, or generated by sound, light and/or radio frequencies. The invention cover the use of whole cyanobacterial algae or extracts thereof, with different levels of transdermal penetration, be it the low intensity transporation, which can be used for deep aesthetic and skin regenerating purposes, as well as dermatological problems; or the high intensity transporation, able to penetrate deep though the dermis, and beyond the hypodermis into organs and tissues, and that can be uses for muscular, osteo-articular and metabolic problems. Following an exhaustive explanation of the invention, we present very significant results in different beauty and health areas.

### BACKGROUND OF INVENTION

The idea of inserting a substance in a specific part of the human body for therapeutic purposes starts with the invention of the syringe with a needle by the French surgeon Pravaz in 1840. Afterwards, there have been many researches and attempts to find ways that could be less traumatic and more efficient. One such system, for instance, is mesotherapy, which still utilizes the syringe idea, but uses a certain number of micro-needles. The first non-syringe related transdermal technology has been ionophoresis, which uses an electrical continuous current to administer transdermally ions of different pharmaceutical substances. This original method has then been improved upon by such method as iontophoresis, a form of electroporation; or even ultrasound technology, which uses variable frequencies ultrasounds to create an energy impulse capable of opening up the outer strata of the skin, helping molecules to go through. Recent studies have shown for instance how iontophoresis has the same ability to promote the passage of high molecular weight substances through the human sclera (Nicoli S. et al, 2009), while the same has been claimed for systems combining different methodologies (Mitragotri S., 2000).

Different methodologies and equipment can be used to push whole molecules, even large, through the skin or the dermis, down to even tissues and organs, and such system have expanded to include also radio frequency (RF); vibration and cavitation by sound; phototherapy by laser or LED light; and even the application of thermal energy. However, the aesthetic or therapeutic result of such enterprises do not depend solely or principally on the technique used, but preeminently on the type of molecule used and on its therapeutic effectiveness.

Cyanobacteria algae are the source of many both nutritional and nutraceutical molecules, endowed with antioxidant anti-inflammatory, anti-proliferative, neuromodulating, neuroprotective, immune-enhancing and so on (as we shall see in the course of the presentation). We have already developed two patents on extracts from Klamath AFA algae, one on its neurologically active molecules (WO2008/000430), and another one on its special phycocyanins and their antioxidant, anti-inflammatory and anti-proliferative use (WO2008/000431). However, none of those patents, nor other patents, have ever considered the transdermal use of the whole cyanobacterial algae or of their phycocyanins, or of other antioxidant, anti-inflammatory and anti-pain molecules, such as phenylethylamine (Mosnaim A D et al. 2014), contained in them.

In our novel experimentation, we have found for instance that the transdermal use of the Klamath microalgae and of its phycocyanins extract, alone or in synergy with others active ingredients (including Spirulina's phycocyanins extract), generates better and faster results than those obtained using other and possibly more well-known nutritional and pharmacological molecules, as will be shown in the next clinical and factual examples.

### THE INVENTION

The invention refers to specific ways to transdermally vehiculate cyanobacterial algae or extracts thereof, such as through electroporation, sonoporation (sound), photoporation (light), radioporation (radio frequency) cavitation, and any other transporation tool which can enhance the transdermal penetration of cyanobacterial molecules. Transporation is achieved via the transmission of some force, be it electric, or generated by sound, light and radio frequencies. These systems, which differ from generic transdermal patches, which are based simply in prolonged contact, use some type of force, be it electrical or other, to promote deeper skin penetration by opening the water and ionic channels within and outside the skin's cells. Such systems can also modulate, via the intensity of the force and the time of the application, the depth of the penetration, achieving a partial penetration of the substance vehiculated down to the dermis, to affect for instance elastin and collagen fibers when using low level transporation intensity or shorter times; while reaching down trough the dermis and into the hypodermis, to affect organs and tissues, via higher intensity and longer time transporation. In this way, these transporation devices allow for the penetration of high molecular weight cyanobacterial molecules such as phycocyanins or large cyanobacterial carotenoids, in order to alleviate and possibly resolve both superficial, epidermis-related beauty and dermatological problems, and deep-rooted skin problems, as well as muscular and bone affections, and possibly even metabolic and organ pathologies.

If we consider for instance phycocyanins, the typical cyanobacterial molecules that have known antioxidant (Bath V. B. et al. 2000; Benedetti S. et al. 2004; 2010), anti-inflammatory (Hsiao G et al., 2005; Romay C. et al, 1998) and anti-proliferative (Scoglio S. et al, 2016) properties, when applied cosmetically, or even applied through transdermal patches, they can only operate superficially. What makes transporation different from mere cosmetics cremes is that it allows for the penetration of the epidermis down to the dermis, to affect for instance elastin and collagen fibers, and even through the dermis and into the hypodermis with enough concentration as to effectively affect muscles, bones or organs. Our previous patent (PCT/EP 2007 005623/published as WO2008/000431) also covered the cosmetic use of AFA-phycocyanins (the name we use for the specific phycocyanins of Klamath microalgae). However, through cosmetic cremes or transdermal means such as transdermal patches, phycocyanins could not go very deep through the skin, and could have mere cosmetic effects; while with transporation, at low intensity levels they can reach the dermis, affecting elastin, collagen and the connective tissue supporting the skin; while with high intensity transporation devices they can reach through the dermis and, via the hypodermis, the underlying organs and tissues, such as muscle, bones, and the bloodstream.

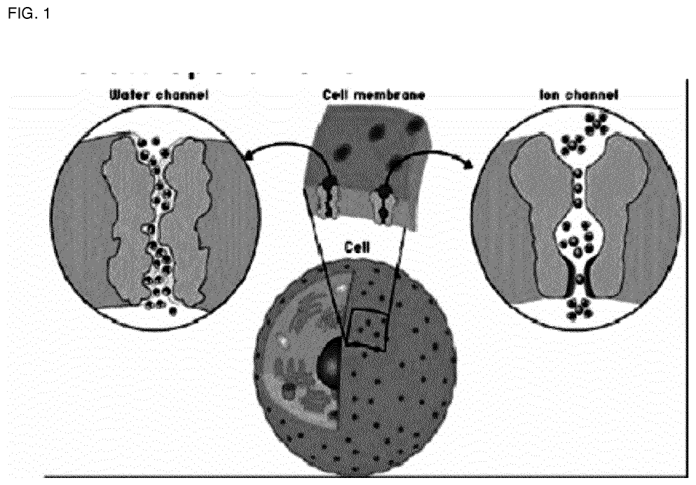

The fundamental problem solved by such transdermal means it's bypassing the resistance of the stratum corneum of the our skin: the phospholipidic intercellular belayer of the skin is naturally hydrophobic; and only the long standing research that led to the discoveries by MacKinnon and Agre (Nobel prize in Chemistry in 1993—FIG. 1), showed that cells have specific and selective channels that allow for the passage of both water and ionic salts. Essentially both low and high intensity transporation work on the same principle of opening up the water and ionic channel in the lipid hydrophobic layer, their difference being only in terms of the depth of penetration of the molecules into and through the skin.

As the process consists in the temporary stimulation of an existing physiological structure and function, the process itself is absolutely physiological, and thus generally safe. However, it is a form of artificial modification of the state of the epidermic cells's water and ionic channels, and this is what distinguishes transporation methods, in its different forms (electro, sono, photo, RF, thermal), from mere transdermal cosmetic means, such as transdermal patches, which do not modify the status of water and ionic channels of the skin's cells, and are thus limited to the transdermal vehiculation of only small molecules, characterized by a molecule size and weight small enough so that the skin does not constitute a barrier for them. So, for instance, molecules such as phycocyanins, which are quite large, or even whole cyanobacterial algae, are too big to penetrate into and through the dermis by themselves.

Once applied through trans-poration methods, phycocyanins, as well as large anti-oxidant and anti-inflammatory molecules contained in the microalgae, such as the AFA-phytochrome described in the above mentioned patent (WO2008/000431) with a molecular weight of approximately 400 kD, can directly and positively affect deep aesthetic and dermatological, as well as muscular, osteo-articular, circulatory and even metabolic diseases, thus bypassing the issue of metabolization through the digestive tract, and throughout the circulatory torrent, which indeed significantly reduces the ability of large, high molecular weight, molecules to arrive to the local, peripheral site where they are needed.

The invention, thus, does not refer to any specific technology or machinery, but to the fact that any result obtainable with the different transporation devices available, is radically enhanced, both in terms of efficacy, speed and cost, by using as the main substance to be vehiculated cyanobacterial algae, such as Klamath algae (Aphanizomenon flos aquae) and/or Spirulina, and/or any of the cyanobacterial antioxidant and anti-inflammatory molecules concentrated in any cyanobacterial extracts.

Other substances can be added to the whole cyanobacteria and/or cyanobacterial extracts, such as phenyethylamine, hyaluronic acid, herbs, specific minerals, enzymes, and so on, but always using the whole cyanobacterial microalgae and extracts as the main component of the products.

The invention consists thus in the transdermal use of a powder, solution, gel or liquid, composed of the whole cyanobacterial algae, be it Klamath AFA, Spirulina or any other, by itself or together with any cyanobacterial extracts concentrating the antioxidant and inflammatory substances such as phycocyanins, phenylethylamine (PEA), MAAs, polyphenols, carotenoids, chlorophyll (Kamat J P et al, 2000; Hartigg U. 1998), etc., to be used trans-topically via different transdermal and trans-poration systems, for health and beauty purposes, through devices that are able to open up the water and ionic channels of the skin. Some of these molecules, such as MAAs and PEA, are quite small, but being embedded in the whole cyanobacterial matrix, they follow the destiny of the larger molecules contained in the matrix.

The preparation can be constituted also by a mix of different cyanobacteria or cyanobacterial extracts, and can be put into bottles, sachets, or any other containers.

Even though the invention implies the possibility of using each cyanobacterial algae and each cyanobacterial extract by itself, the preferred preparation is indeed a mix of both the whole cyanobacterial algae and the cyanobacterial extract, such as the one described in the patent WO2008/000431. This is because, while the cyanobacterial extract concentrates some of the most powerful antioxidant and anti-inflammatory substances contained in the microalgae, the whole microalga itself contains further components, such as vitamins, minerals and other nutraceutical molecules, that can add a significant further action to the vehiculated preparation.

For instance, cyanobacterial algae contain some trace minerals that may not be very effective when ingesting the algae orally, given their dispersion through the overall body metabolism, and the possibility that they may not be able to cross the intestinal membrane; but that, when vehiculated transdermally, they may actually reach the site that needs to be helped and healed much more directly. A relevant example is that of boron, a trace mineral that can be very helpful for the fixation of calcium in the bone, and thus for osteoporosis, and that is present in Klamath algae at a concentration of about 11 mcg./gr., a low quantity in terms of recommended daily intake, and that with the algae taken orally would be relatively ineffective, when we consider that the few micrograms present would be dispersed throughout the body at the level of naonograms, if not picograms. However, even just 11 micrograms, when administered via transporation onto the area affected, can be very relevant and effective.

This is even more true, when we consider that Klamath algae contains about 15 milligrams/gr. of calcium, a dosage that taken orally represent only a small fraction of the daily requirement, but if injected directly into the area covering the affected bone, can actually be very helpful to all osteo-articular and bone problems.

The same can be said of : a) essential fatty acids, such as Omega 3 or Omega 6, present in all cyanobacterial algae, but at a level that, when considered in relation to the oral dose, is still relatively low, whereas it become very effective when applied locally through a transdermal device; b) antioxidant vitamins such as the vitamins C, E and some vitamins B, that can be present in small quantity not very effective hen taken orally, but very useful when injected transdermally; c) many trace minerals present in the algae (AFA algae contains more than 70 minerals and trace minerals), which when taken orally may have a too low concentration to be effective, but when injected transdermally directly in the treated area, they actually can be very effective; d) cyanobacterial algae are also rich in carotenoids, chlorophyll, polysaccharides and polyphenols; but given the low oral dosage (3-5 grams), again their concentration is relatively low (except for a few nutrients), but again it becomes very effective when applied locally, thus without systemic dispersion, via transdermal devices.; e) AFA-algae is also rich in phenylethylamine, a molecule endowed with specific neurological, immunological (Babusyte et al. 2014) and analgesic (Mosnaim A D et al., 2014) properties.

Given the nutritional and nutraceutical richness of cyanobacterial algae, such as Klamath algae or Spirulina, they can generate an optimum synergy with any other phytotherapic and nutraceutical substances. In fact, any single molecule is usually in need of some other molecule playing a supporting role, and in this respect cyanobacterial algae cam provide the best synergy possible. This is why, for each different area of intervention, the cyanobacterial algae, constituting the essential base, can be combined with other substances, each one relevant for the specific area of intervention.

Therefore, the invention also covers not only the transporation vehiculation of cyanobacterial algae and of their antioxidant and anti-inflammatory extracts, but also the synergic use of cyanobacteria with any other substance able to intervene favorable on the specific area to be treated. Such use of the whole cyanobacterial algae and/or their extracts in association with other synergistic ingredients, can be applied to the following areas:
A) Aesthetic, including problems such as wrinkles, cellulite, stretch marks, age spots, muscle and skin tone, breast volume, etc.
B) Dermatological, including eczema, psoriasis, wounds, scars, etc:
C) Osteo-articular, including arthritis, inflammation of the joint, joint calcifications, inflammation and/or calcifications of the tendons, degeneration of cartilage such as meniscus, osteoporosis, and so on;
D) Muscular, such as muscle lesions, traumas, inflammation and muscle degeneration;
D) Metabolic, such as lymphatic (diabetes, aging) and circulatory problems (varicose veins, heavy legs, etc), obesity, fat metabolism, and so on;

We shall see how the application of products based on Klamath algae and Spirulina as the preferred cyanobacterial algae can have very effective results when associated with specific ingredients in each specific area of application.

### Areas of Application in Detail

AESTHETICS—In this area we tested the transdermal use, with low voltage electroporation, as well as low frequency sonoporation and RF, and low intensity light, of a product composed of the whole Klamath algae, of Klamath algae's and Spirulina's phycocyanins extracts, combined with different other substances for each specific purposes. The main conditions tested are the most difficult to treat in the field, namely wrinkles, cellulite, stretch marks, tone of skin, tone and volume of breast, scars and keloids, wounds and so on; all conditions that do not require any penetration through the skin and into organs and tissues.

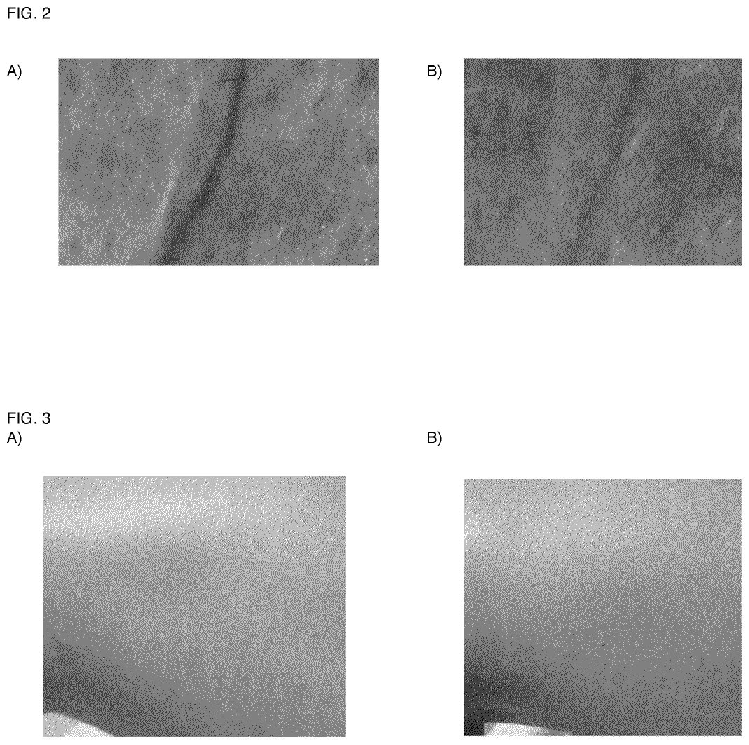

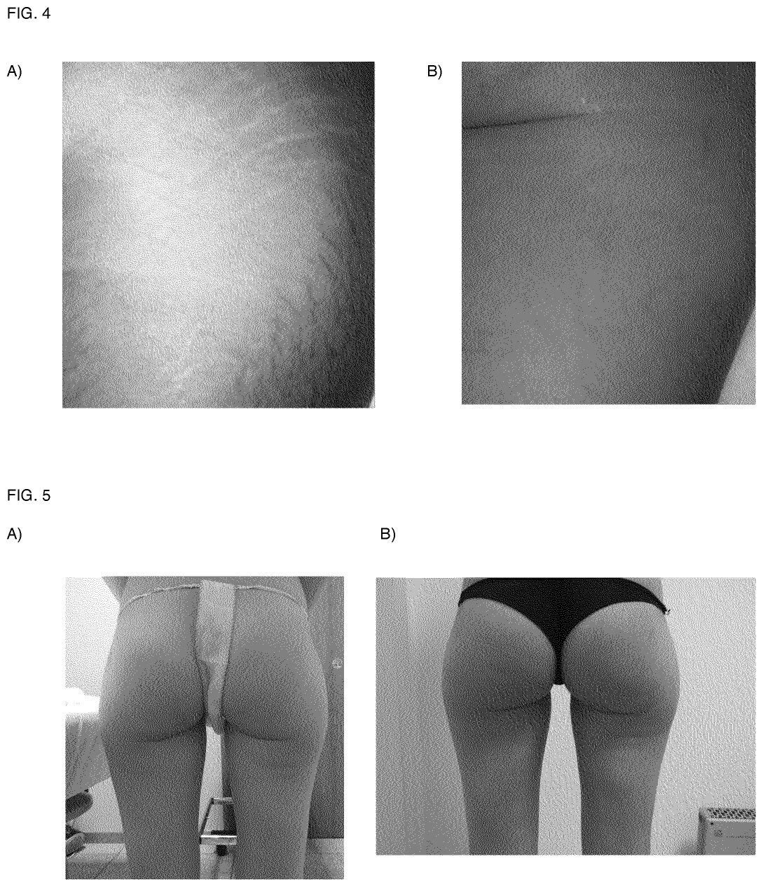

While for tonification and filling purposes, as wrinkles, stretch marks and wounds we only added, to the main cyanobacterial base, high molecular weight hyaluronic acid; for breakdown purposes, in conditions such as cellulite and keloids, we also added some herbal extracts as well as proteolytic and lipolytic enzymes.
Wrinkles—Wrinkles are mostly the result of aging, and are due to the breakdown of collagen emerging with age, and more specifically with the increase in oxidative and inflammatory processes, of malnutrition that does not provide the body with enough antioxidants and protective nutrition (vitamins, minerals, EFA), and a lowering of the body's ability to regenerate. To respond to this aging phenomenon, different strategies have been used: providing antioxidant and anti-inflammatory molecules to the skin; supplying the skin with missing nutrients; stimulate the skin's ability to regenerate. Cyanobacterial algae allow all these strategies to be used at one time: they contain a wide range of bioavailable nutrients, have a broad spectrum of antioxidant molecules; their phycocyanins have a high anti-inflammatory power (5); finally, recent studies have shown that phycocyanins potentially stimulates the activity of fibroblasts, endogenous molecules that act as collagen and connective tissue precursors, and so are essential for skin and dermis to regenerate (9). Thanks to all these properties, we found that the transdermal application, via low voltage electroporation of a product containing cyanobacterial algae, the whole Klamath algae, Klamath and Spirulina extracts, plus high molecular weight hyaluronic acid, produces profound and rapid results. Here we report a single case example where, in order to verify more specifically the ability to work on wrinkles, we decided to use a high-definition camera to photograph a single very obvious wrinkle before and after treatment in a woman. [FIG. 2]
Stretch marks—Stretch marks are a form of scars on the skin, caused by dermatitis. They can be due to rapid skin stretching associated with rapid recurrence (common in puberty) or weight gain (i.e., pregnancy or muscle mass increase). Stretch marks may also be affected by hormonal changes associated with puberty, pregnancy, and muscle mass increase. The main physiological cause of stretch marks is the fact that glucocorticoid hormones affect the epidermis by preventing the action of fibroblasts forming collagen and elastin fibers. This creates a lack of support material, as the skin is pulled and leads to dermatitis. Phycocyanins have been shown to be potent fibroblast activity stimulants, and we have already shown how Aphanizomenon flos aquae (AFA) algae contains a very powerful form of phycocyanins (6, 7), which is thus preferred, even though it can be combined for synergic purposes with Spirulina's PCs and other possible ingredients. Phycocyanins can thus be very beneficial in treating stretch marks, especially if introduced directly into the dermis by low intensity electric, sonic photo and RF transdermal vehiculation. We have experimented with various stretch marks by applying onto the affected areas the whole AFA algae, an AFA-phycocyanins as well as Spirulina's PC extracts and high molecular weight hyaluronic acid, through an electroporation+sonoporation machine, and remarkable results have been obtained, as shown in the case reported in FIG. 3; as well as in the case of a woman of 38 years with obvious stretch marks in her legs, which have almost disappeared after just 3 treatments (FIG. 4).
Skin tone—The right skin tone depends on the structural integrity of the dermis and its essential components, such as collagen and elastic fibers as well as the underlying connective tissue. Fibroblasts are precursor cells of all such components, and the ability to properly activate fibroblasts plays a central role in maintaining the tone and integrity of the skin. Phycocyanins are powerful stimulators of fibroblast and fibroblast activity (Madhyastha, H. K. et al., 2008; 2006) and in that sense seem to be potentially very useful in restoring and maintaining the correct skin tone. Our experiences through the transporation of cyanobacterial algae and phycocyanins extract have confirmed these hypotheses.

We have tested some cases to see if the transdermal vehiculation of a product comprised of the whole AFA plus AFA-phycocyanins and Spirulina's phycocyanins extracts, plus some high molecular weight hyaluronic acid, can improve skin tone in humans, and the results have bee astounding, as shown in the following cases:

a) The case of a 35-year-old woman whose buttocks showed a tendency towards decreasing dermal tone, which was completely re-established after only 3 treatments (FIG. 5);

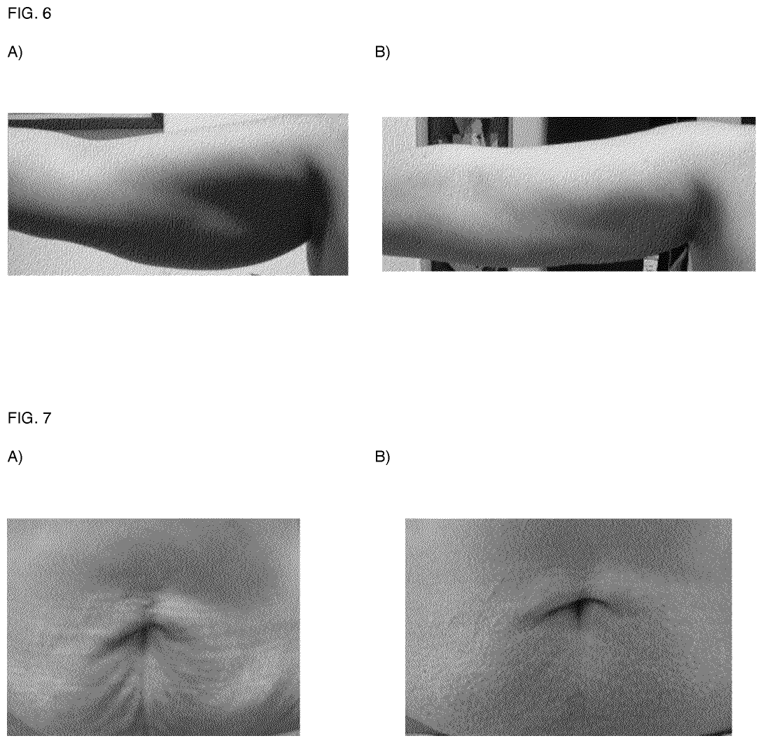

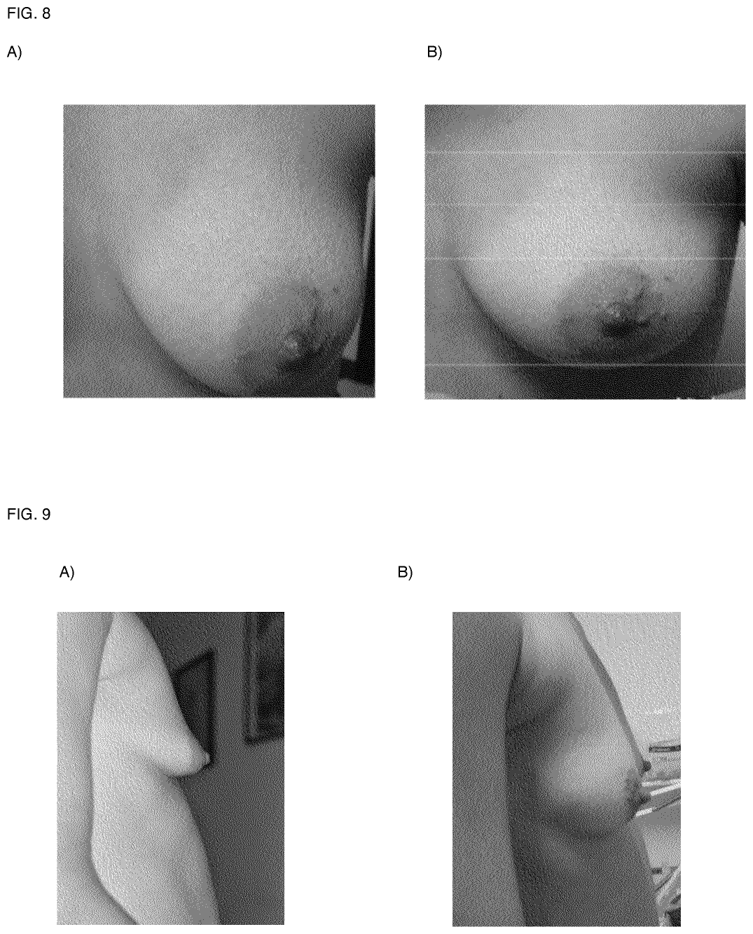

b) the case of a 47-year-old man with sagging skin in his arms, whose tone was completely re-established after only three treatments (FIG. 6); c) a 52 year old woman, whose belly's skin was deeply sagging, and whose tone was very significantly restored after only 1 treatment (FIG. 7).
Breast tone and volume—The logic behind the effectiveness of the cyanobacterial transporation in relation to the tone of the female breast is similar to one already discussed above concerning collagen and elastin. Regarding breast volume, this may depend on other factors, which at present are still unknown. Certainly the fibroblasts stimulation generated by the phycocyanins can play a role. Though we obtained significant results on breast volume with the usual product containing whole AFA, AFA and Spirulina's phycocyanins extracts, as well as high molecular weight hyaluronic acid, it is possible that adding to the product isoflavones from vegetable sources, given the hormonal component of breast structure, could further improve our results. As to breast tone, we report here one of the most significant cases, a 22-year-old woman with slightly drooping breasts, whose breasts tone increased very clearly after 4 sessions (FIG. 8). As to breast volume, we report the case of a young woman with a very small breast, which increased in size quite significantly after 8 treatments (FIG. 9).
Cellulite—When we move to cellulite, the situation becomes more complex, as it involves various factors, such as inflammation, fluid retention, lymphatic and venous stagnation, hypertrophy of fatty tissue, with each factor playing a different role in each individual case.

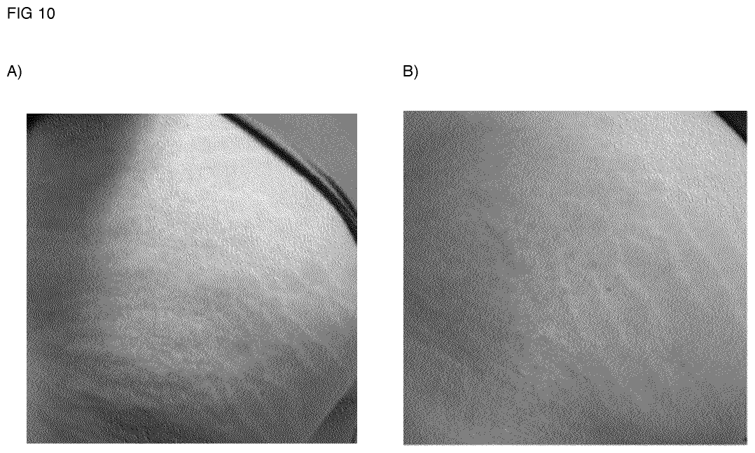

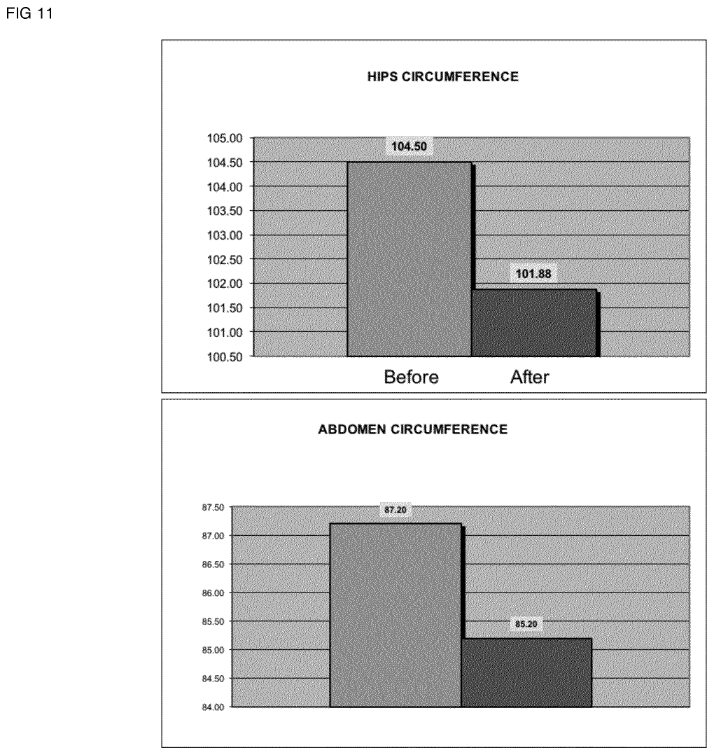

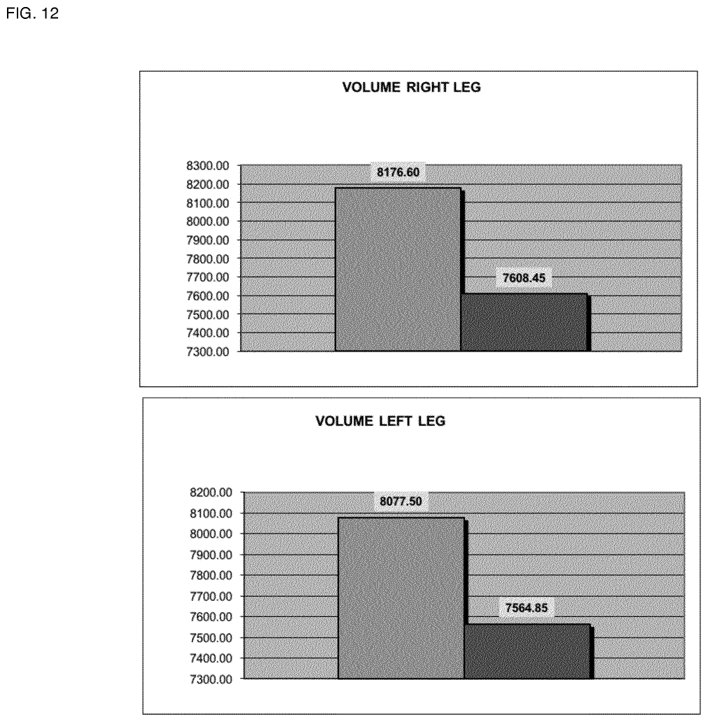

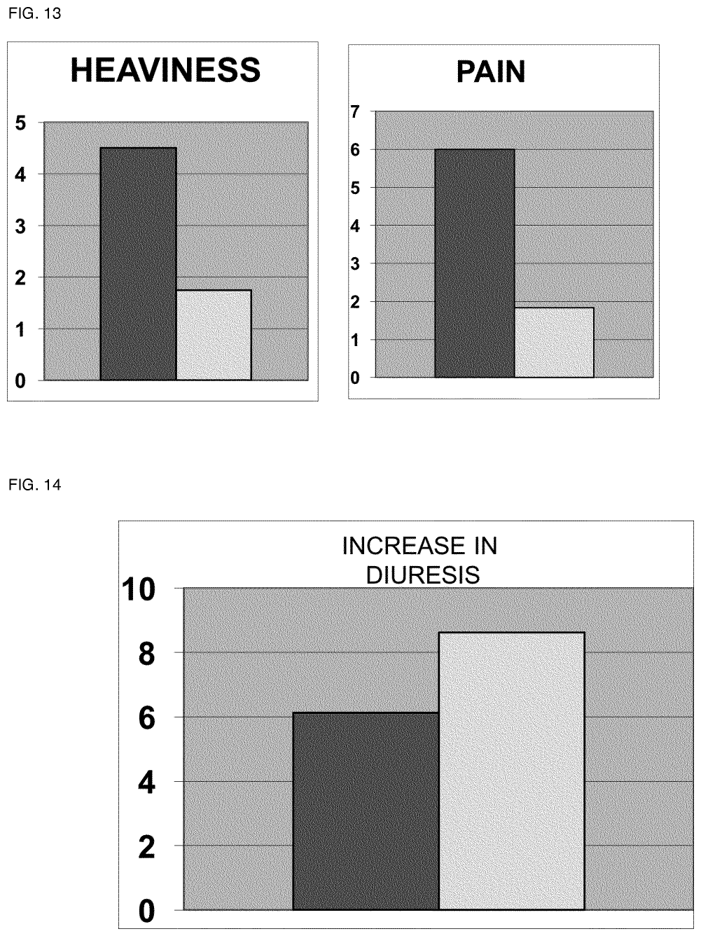

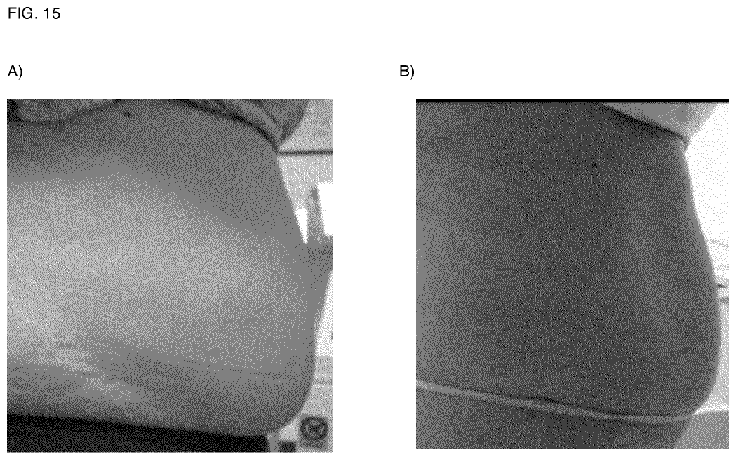

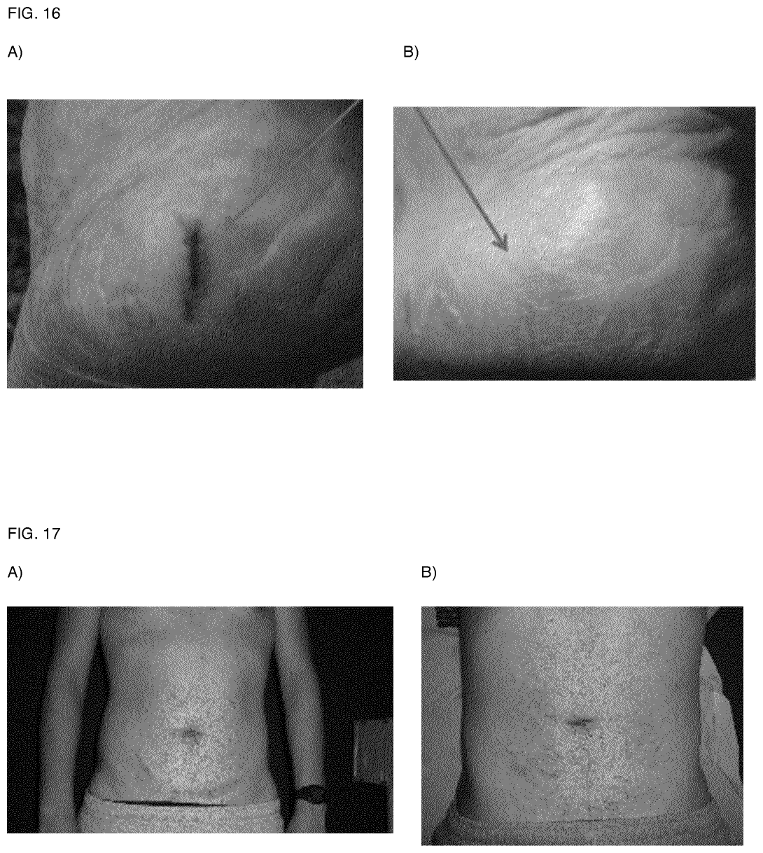

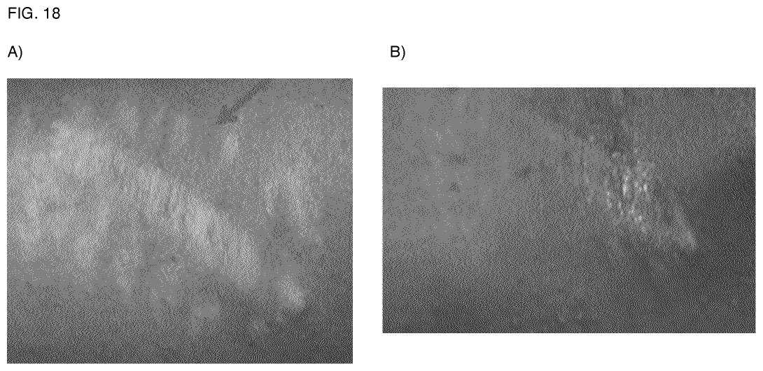

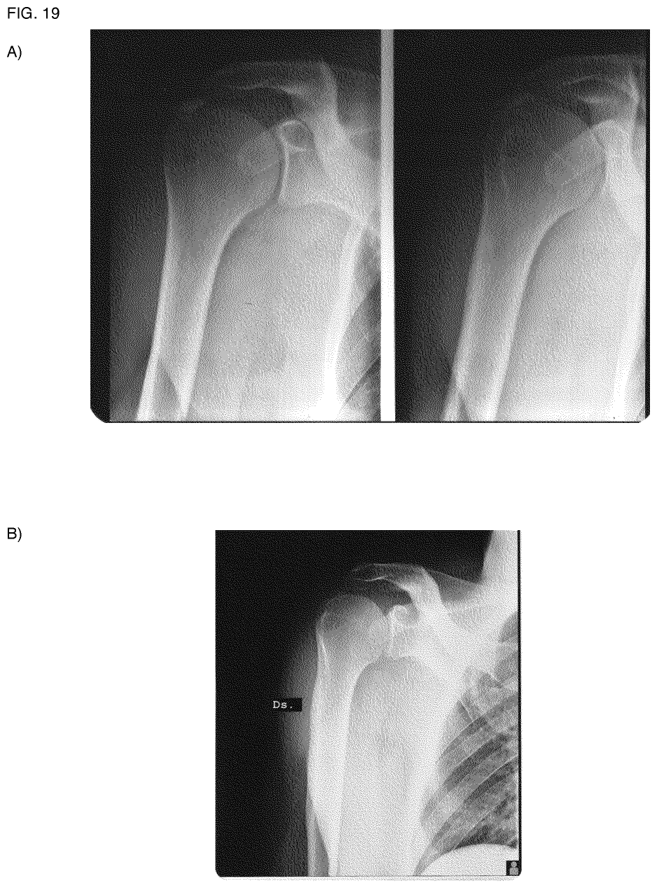

The product we used in this case, while having at its core still cyanobacterial algae (AFA, Spirulina) and relative extracts, added as complementary ingredients proteolytic and lipolytic enzymes and herbs. The basis for the efficacy of this product is once again based on the antioxidant and anti-inflammatory activity of the active molecules present in cyanobacterial algae. In particular, phycocyanins have been shown to be active also as anti-platelet aggregation factors, and platelet aggregation plays a role in both venous and lymphatic stagnation (8). Also, phycocyanins have shown to have a significant fibrinolytic activity, which is essentially to break down the fibrin that holds up the the cellulite debris together (9). Finally, both proteolytic and lipolytic enzymes are also fibrinolytic, and thus again capable of dissolving the fibrin that holds cellulite tissue together. We have regularly tested the resolution of cellulite cases with the transdermal application of a cyanobacterial based product. Here we present the case of 54-year-old woman with a type 3 cellulite, who received a significant improvement after only three treatments during the same week (FIG. 10). More importantly, we have done a study at a medical center in Italy, in which subjects underwent only 3 transporation treatments, and whose symptoms associated with cellulite were evaluated:
a) circumference of abdomen and hips, which is known to increase with the accumulation of cellulite on the tissues (FIG. 11);
b) the volume of the legs affected by cellulite, which again is shown to increase with cellulite and fat deposits (FIG. 12);
c) the sense of heaviness and pain associated with the cellulite (FIG. 13);
d) the increase of the diuresis (“aumento diuresi”) associated with the break down and elimination od cellulite deposits (FIG. 14).
Localized adiposity (fat belly and fat thighs)—Localized adiposity is associated with bad fat metabolism, and fat accumulation in local tissue, especially around the waist. In addition to the positive effects that can be created by diet-based systemic intervention, transporation in the area with excessive adiposity with a cyanobacterial product, plus certain specific ingredients such as lipolytic enzymes, can be very effective. Here we have used a higher intensity transporation, that is higher voltage electroporation. In fact, fat tissue rests underneath the dermis, and so a deeper penetration is required to help dissolve and mobilize accumulated fat. The cyanobacterial algae and extracts seems to be able to intervene in the synthesis of fat, reducing the production of arachidonic acid, and hence the inflammation and oxidation of fats behind the accumulation of localized fat (Kushak R. I. et al., 2000). Phycocyanins are also accelerators of metabolism, due to their ability to counter platelet aggregation (Hsiao G. et al, 2005), thus improving blood circulation and ability to remove fat deposits and debris in various tissues. We tested the transporation of this formula on various cases of abdominal and thighs fat accumulation, with rapid and profound results and with obvious reduction of the belly and legs circumference, as shown in the following cases:
   a) a 38-year-old woman who wanted to reduce the circumference and volume of her thighs, which was visibly achieved, with minus 2.5 centimeters circumference reduction, in only 2 sessions (FIG. 15);
   b) a 52-year-old woman, whose belly and waist circumference was significantly reduced after only 6 treatments (FIG. 16).
DERMATOLOGY—In this area we have experienced excellent results through transdermal application of a product containing cyanobacterial algae and their anti-inflammatory extracts, in conditions such as eczema, psoriasis, various forms of dermatitis, acne, as well as healing wounds and scars.
Psoriasis, eczema and acne—The cyanobacterial algae, and especially the AFA and Spirulina's algae phycocyanins extract, play an important role here, since all these conditions involve some forms of skin inflammation. Indeed, in a previous study our research group showed that even just oral intake of a product containing a significant amount of AFA-Phycocyanins could produce good results on various types of psoriasis (Cavalchini & Scoglio, 2009). Low intensity cyanobacterial transportation has shown to significantly strengthen this activity. We recorded: a) two cases of localized psoriasis: in one case, a psoriatic eruption in an area around the mouth, completely resolved in two sessions; while long-term psoriasis in the scalp was solved with three sessions; b) two cases of eczema: in one case, an eczema that afflicted a 16-year-old male for more than three years was completely removed after only 2 sessions; in another case, a woman with an old-time psoriasis in her hands had a significant improvement already after a session, and a complete disappearance of localized psoriasis after 3 more sessions. Finally, we have also tested two cases of acne: in the first case, a serious case of facial acne has been considerably improved with 4 sessions; in the second case, a moderate type of acne has been completely eliminated within 2 sessions. All cases were tested by transdermal vehiculation of a product containing: Klamath algae and Spirulina's phycocyanin extract; proteases and lipases.
Scars and wounds—Once again, the strong ability of phycocyanins to stimulate fibroblasts is very important, especially in the healing of wounds, to the point that they have actually been proposed as possible remedies for ulcers and other types of wounds (Madhyastha H K. et al. 2006). We tested a case of diabetic foot ulcer, and with only three sessions we were actually able to fully heal the chronic wound (FIG. 17). As for the scars, we have had enough positive cases with a product that added, to the basic cyanobacterial content (AFA algae and phycocyanins extract+Spirulina's phycocyanin extract), also proteolytic and lypolitic enzymes and some special herbal extracts, such as curcumin and caffeine. Here we present two of those cases:
   a) a 30-year-old man with a long, large scar at the right side of his abdomen had a significant reduction in both visibility and scar thickness only after four sessions, during which he also had a parallel reduction in the size and volume of his belly (FIG. 18);
   b) a keloid, resulting from a long standing scar, which significantly and visibly reduced after 5 sessions with the transdermal application of the above indicated product (FIG. 19).
MUSCULAR TRAUMAS, LESIONS AND INFLAMMATIONS—In this area, again, phycocyanins can play a very important role, both for their anti-inflammatory and anti-edema power, as well as for their trophic and regenerative properties, via fibroblasts stimulation. However, when moving to muscles, we need to use high intensity transporation, such as higher voltage electroporation or high frequency sonoporation.
Muscle lesions/traumas—With reference to muscle lesions and traumas, the ability of phycocyanins to inhibit COX-2 and lipoxygenase enzymes, as well as their ability to modulate factors such as TNF-alpha, is very important with reference to the need for a rapid reduction of inflammation and associated pain. Muscle trauma can be divided into direct (bruises) and indirect injuries (stretches and tears). The latter are obviously more serious and difficult to cure, especially if they are 2nd degree injuries. Typical of athletes, these indirect muscle injuries can hold an athlete of any sporting activity (or any other human being unable to walk regularly) for weeks. We have tested four cases of indirect muscle injury:
   a) the first case concerns a 19-year-old soccer player with an indirect type 1 injury sustained during the a Sunday game. He was treated Tuesday and Wednesday and the pain had already gone away; was then treated again on the following Friday and Saturday, and the injury appeared completely healed. He did not play the next game just as a precaution;
   b) the second case is that of a 45-year-old lady with a chronic calf inflammation that did not allow her to move regularly. Four sessions had been scheduled; but after the second session the calf was already fine. After checking her condition three months after the second treatment, the lady continued to be well and had no problems;
   c) the following two cases refer to two soccer players with a 2nd degree injury; in either case the injury was pronounced. The two players were treated mid-week for 3 times (Thursday, Friday and Saturday); by Friday they felt healed, and again they did not play just our of precaution.
OSTEO-ARTICULAR AREA—In the osteo-articular area we are concerned with problems ranging from arthritis, to inflammation of tendons and joints, from joints and tendon calcifications to cartilage degenerations (menus, etc.) to osteoporosis. Once again, the anti-inflammatory properties of phycocyanins are paramount, and in cyanobacteria they work in synergy with its other anti-inflammatory molecules, such as chlorophyll and polyphenols. But the ability of phycocyanins to stimulate the release and the activity of fibroblasts, and possibly of other blasts (stem cells), seem to play an essential role in promoting regeneration of cartilage, bones and muscles. Here too we need to use high intensity transporation, and found that such transporation significantly improves the action of the cyanobacterial extracts, achieving much faster and deeper results.
Inflammation/pain of joints and tendons—We have experienced three serious cases of inflammation of the joints and tendons:
   1) a case involving a professional karateka athlete whose knee was badly compromised and for this reason had to stop training; the knee was treated twice with the transporation of a product including cyanobacteria algae and extracts, as well as proteolytic enzymes and high molecular weight hyaluronic acid, which adds an acton of its own in the regeneration of cartilages. After only 2 treatments, the athlete was free of pain and inflammation, and after 2 more treatments he returned to training and competing regularly;
   2) a similar case involved another karateka, which had a wound to the right tendon and which for this reason had stopped exercising and competing for a few months. He was subjected to a treatment with the same product as above. After only three treatments, the athlete was free of pain and inflammation, and went back to training and competing regularly;
   3) an elderly man with a large cyst behind his knee, with pain, inflammation and inability to completely bend his knee. The man was treated transdermally with the same product above and immediately after the first treatment, there was a marked reduction in the volume of the cyst, which passed from the size of a big lemon to that os a cherry kernel, plus the complete disappearance of the pain, and with the recovered ability to extend almost completely the knee.
Joints and tendons calcifications—Calcium deposits tend to form around the joints and tendons, for reasons that are not too well understood. At first they are mild, but over time they tend to harden and grow to the point of generating constant inflammation and consequent pain. There is really no treatment, except for a temporary anti-pain injection and the use of so-called “shock waves”, which however have limited efficacy, some side effects and complications, and a relatively high cost. In fact, in most cases the only solution that is offered is the surgical removal of the stones (calcium deposit) and this also has its problems because, apart from the invasive effect of the intervention, the recovery and rehabilitation periods are long and expensive. And there is no guarantee that calcium deposits will not form again. We have found, after numerous experiments, that the high level transporation of a product based on AFA alga and AFA-phycocyanins extract, plus Spirulina's phycocyanins extract, and some more specific ingredients that we shall discuss, can be extremely effective in this field. The first case we treated was done with the usual cyanobacterial ingredients plus proteolytic and lipolytic enzymes, and there was not only a radical pain and inflammation reduction, but also an unexpected positive result on calcification. It was the case of a lady who suffered from chronic Achilles's tendon degeneration, with advanced calcification and inability to walk properly. His specialist doctor had already set the appointment for a surgical calcification removal. The patient underwent four sessions with the transporation of the product described above. After four sessions the pain had completely gone away, she returned to walk regularly, even with high heels, and thus canceled the operation. At the same time, the lady also made a magnetic resonance that showed how calcification had been reduced by more than 60%. We have therefore deduced that the AFA algae and phycocyanins extracts, in synergy with specific enzymes, can promote the dissolution of calcium deposits. This may in fact be mediated by anti-inflammatory action, being known that calcium deposits are essentially modulated by inflammation. The anti-inflammatory action of the molecules contained in the algae has been shown to be useful also in reference to this problem, as all the test patients reported significant improvement in pain and mobility, even after the first treatment.

At this point, however, we decided to develop a more specific formula for such a painful and widespread problem, that has currently no satisfactory solution, adding to the product described above a mixture of specific minerals, endowed with the ability to generate the lysis and dissolution of calcium deposits. The list of the minerals added to the cyanobacterial formula includes, without being exhaustive, acetic acid, citric acid, EDTA (ethylenediaminetetracetic acid), sodium bicarbonate and sodium hydroxide.

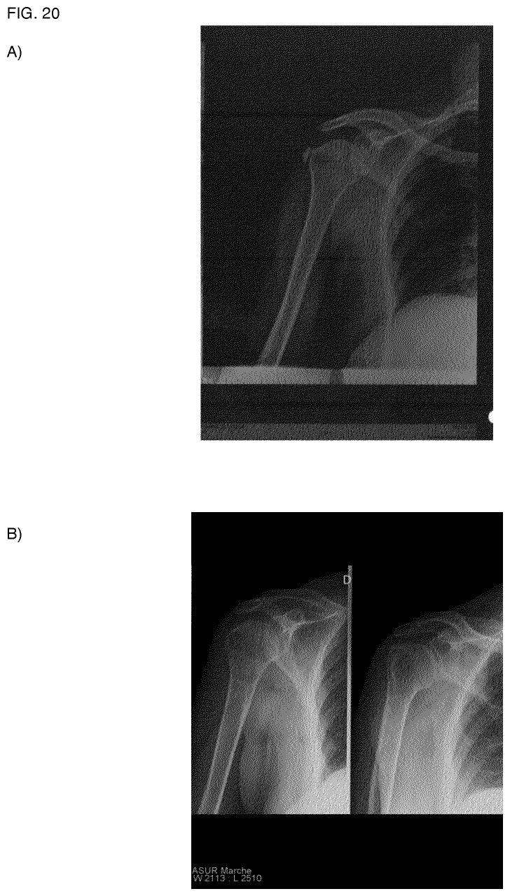

This formula has been tested, the main ingredient still being the cyanobacterial algae and extracts, plus enzymes plus the above mentioned minerals. We have tested it in over 50 cases of more or less advanced calcification of the joints. Each of the participants in the study was asked to perform an ultrasound scan before the treatment, to evaluate the position and size of the calcification. Each participant with a proven calcification was then treated with the transporation of the above mentioned formula, and the result was verified through a further post-treatment ultrasound scan. In all 50 cases, on average after only four sessions and sometimes after 6 sessions, calcifications were completely dissolved, and the patients completely recovered the mobility of the joint. We believe that the transporation of the above-described formula can be an effective therapeutic solution of a previously unresolved problem that afflicts an ever-growing segment of the population. Here we report only two of the 50 cases that were recorded:
a) a 38-year-old man with a 15 mm (1.5 cm) long shoulder calcification, that was completely removed after four sessions of the trans-poration of the same formula, with the patient recovering the full arm and shoulder functionality (FIG. 20);
b) a 65-year-old woman who had developed a calcification of her shoulder many years before, which hindered her function and constantly caused pain; she was treated with the trans-poration of the formula described above, and the calcification was completely dissolved (FIG. 21);
METABOLIC—We did some tests with interesting results on metabolic dysfunctions such as lymphatic congestion (due to diabetes or aging) and circulatory problems (varicose veins, heavy legs, etc.). Phycocyanins (AFA-PCs or Spirulina PCs) also have anti-platelet aggregation activity, and thus contribute to adequate blood flow, thereby counteracting excessive blood density which is also responsible, at least in part, for the most common circulatory problems such as varicose veins. In addition, inflammation plays an important role in such problems, and we have already indicated how phycocyanins express their anti-inflammatory action much more effectively if applied in situ through transporation. In addition, circulatory problems are also related to the weakening of the capillary arteries as well as, more generally, the laxity of the parietal membranes of the blood vessels. Phycocyanins have a significant stimulating activity of fibroblasts, which are also involved in the production and maintenance of structures such as collagen, elastin and more generally connective tissue, which are essential for the health and tone of the blood vessels. Therefore, AFA-PCs as well a Spirulina PCs, administered transdermally, can play an essential role in the treatment of vascular problems. Further contributions to the efficacy of the treatment were made with the addition of proteolytic and lipolytic enzymes, which are characterized by both a specific anti-platelet aggregation and fibrinolytic activity. We treated two women, one younger (35) and one older (65), with varicose veins in the legs. Both women, after three treatments the first, after four treatments the second, reported a visible reduction in the amplitude and depth of the varicose veins, claiming to feel much better, with a feeling of leg lightness.
SYSTEMIC—The possibility of achieving significant depths though high level transporation of large molecules, thus potentially reaching through local circulation the inner organs, opens up the possibility of a systemic medical use of the methodology. For example, phycocyanins have a significant anti-proliferative activity (Kunte M. et al, 2017; Ravi M. et al., 2015); and our research group has shown significant anti-proliferative activity for AFA-phycocyanins from Klamath algae (Scoglio S. et al., 2016). It is possible to hypothesize the possibility of affecting, through the high intensity transporation of phycocyanins, organs affected by malignant growth in order to inhibit such growth directly. The same goes for other areas, such as the dissolution of gallstones and kidney stones via the direct transporation on the specific areas.

As a first proof of this hypothesis/possibility, we treated a child with cystic fibrosis, who had already improved his condition through a nutritional supplement program with Klamath algae and different types of fungal enzymes. The parents agreed to submit the child to a series of sessions where the cyanobacterial product (Klamath algae, AFA-phycocyanincs extract, Spirulina's phycocyanins extract), enhanced by proteolytic and lipolytic enzymes, was applied via electroporation to the chest and the back of the child, to see if this could have an effect on the excessive lung congestion affecting the young cystic fibrosis patient. During each of the three sessions, the child expelled a large amount of lung catarrh, interpreted as a positive sign by both physicians and parents. This case preliminarily proves that the transporation of the above described product is able to penetrate so deep as to penetrate the organs, in the specific case the lungs and bronchial tubes.

### REFERENCES

Babusyte A. et al. 2013. Biogenic amines activate blood leukocytes via trace amine-associated receptors TAAR1 and TAAR2, Journal of Leukocyte Biology, 93: 387-394.
Benedetti S., Scoglio S. et al., 2004. Antioxydant properties of a novel phycocyanin extract from the blue-green alga Aphanizomenon Flos Aquae, in Life Sciences, 75:2353-2362.
Benedetti S., Scoglio S. et al., 2010. Oxygen radical absorbance capacity of phycocyanin and phycocyanobilin from the food supplement Aphanizomenon flos-aquae, in J Med Food. February; 13(1):223-7.
Bhat V. B., et al., 2000. C-phycocyanin: a potent peroxyl radical scavenger in vivo and in vitro, in Biochem Biophys Res Commun. 275(1):20-25;
Cavalchini A., Scoglio S., 2009. Complementary treatment of psoriasis with an AFA-phycocyanins product: a preliminary 10-cases study, International Medical Journal, 16 (3), September, pp. 221-224.
Harttig U., 1998. Chemoprotection by natural chlorophylls in vivo: inhibition of dibenzo[a,l]pyrene-DNA adducts in rainbow trout liver, Carcinogenesis, July; 19(7): 1323-6.
Hsiao G. et al, 2005. C-Phycocyanin, a Very Potent and Novel Platelet Aggregation Inhibitor from Spirulina platensis, J Agric Food Chem. October 5; 53(20): 7734-40.
Kamat J P, et al., 2000. Chlorophyllin as an effective antioxidant against membrane damage in vitro and ex vivo, in Biochim Biophys Acta, September 27; 1487(2-3):113-27.
Kunte M., Desai K., 2017. The Inhibitory Effect of C-phycocyanin Containing Protein Extract (C-PC Extract) on Human Matrix Metalloproteinases (MMP-2 and MMP-9) in Hepatocellular Cancer Cell Line (HepG2).Protein J. June; 36(3):186-195:768.
Kushak, R. I. et al., 2000. Favorable Effects of Blue Green Algae Aphanizomenon Flos Aquae on Rat Plasma Lipid, in Journal of the American Nutraceutical Association, Vol. 2, n°3, January, pp. 59-65.
Kushak R. I., et al, 2001. The Effect of Blue-Green Algae Aphanizomenon Flos Aquae on Nutrient Assimilation in Rats, JANA, Vol. 3, no. 4, Winter, pp. 35-39.
Madhyastha H K. et al. 2008. uPA dependent and independent mechanisms of wound healing by C-phycocyanin, in J Cell Mol Med. December; 12(6B):2691-703.
Madhyastha, H. K. et al. 2006. C-phycocyanin transcriptionally regulates uPA mRNA through cAMP mediated PKA pathway in human fibroblast WI-38 cells, in Biochim Biophys Acta. November; 1760(11):1624-30.
Mitragotri S. 2000. Synergistic effect of enhancers for transdermal drug delivery, in Pharm Res. November; 17(11):1354-9.
Mosnaim A D et al. 2014. Analgesic effects of p-phenylethylamine and various methylated derivatives in mice, Neurochem Res. September; 39(9):1675-80.
Nicoli S. et al. 2009. In vitro transscleral iontophoresis of high molecular weight neutral compounds, in Eur J Pharm Sci. March 2; 36(4-5):486-92.
Rafail I. Kushak, et al. 2000. Favorable Effects of Blue Green Algae Aphanizomenon Flos Aquae on Rat Plasma Lipid, Journal of the American Nutraceutical Association, Vol. 2, n°3, January pp. 59-65.
Ravi M. et al. 2015. Molecular mechanism of anti-cancer activity of phycocyanin in triple-negative breast cancer cells, BMC Cancer. October 23; 15.
Romay, C. et al. 1998. Antioxidant and antinflammatory properties of C-phycocyanin from blue-green algae, in Inflamm Res, January; 47(1): 36-41.
Scoglio S. et al, 2016. Inhibitory effects of Aphanizomenon flos-aquae constituents on human UDP-glucose dehydrogenase activity, J Enzyme Inhib Med Chem. December; 31(6): 1492-7.

1. The transdermal vehiculation of cyanobacterial algae, and/or extracts thereof, via electroporation, sonoporation, radio frequency, photoporation, or any other system using some type of force to allow for the opening of the water and ionic channels of the skin, for beauty and health enhancing purposes.

2. The use, according to claim 1, of specific devices, including without being limited to, electrical currents or impulses, such as ionophoresis, iontophoresis, electrical muscle stimulation, and all forms of electroporation; vibratory currents, such as cavitation, ultrasound and all forms of sonoporation; laser, LED light and all forms of phototherapy; all forms of radio frequencies; thermal devices using heat; any other device capable of opening the skin's pores to allow for the penetration, at different levels of depth, of the large molecules.

3. A preparation, according to claim 1, which contains the whole Klamath AFA algae and/or extracts thereof, and/or whole Spirulina or Spirulina's extracts, and/or any other cyanobacterial algae and/or cyanobacterial extracts, combined with any other substances.

4. A preparation according to claim 2, whereby the other substances include, without being limited to, purified phycocyanins, purified phenylethylamine, proteolytic and lipolytic enzymes, hyaluronic acid, glycosaminoglycans, chlorophyll, carotenes, xantophylls (astaxantin, canthaxanthin, zeaxantin, lutein, licopene), polyphenols, polysaccharides, caffeine, vitamins, minerals and herbs.

5. A preparation, according to claim 1, which is formulated in pharmaceutically acceptable vehicles and excipients, wherein all components of the preparation are suitable for administration by transporation methods.

6. The use of a preparation, according to claim 1, applied transdermally via transporation to a subject for preventing, controlling, mitigating or treating aesthetic problems, such as wrinkles, cellulite, stretch marks, age spots, skin and muscle tone, breast tone and volume.

7. The use of a preparation according to claim 1, applied transdermally via transporation to a subject for preventing, controlling or treating dermatological diseases, including, without being limited to, acne, eczemas, keloids, psoriasis, pimples, scars, wounds.

8. The use of a preparation according to claim 1, applied transdermally via transporation to a subject for preventing, controlling, mitigating or treating muscle and osteo-articular diseases such as arthritis, joint inflammation, joint calcification, tendon inflammation and calcification, cartilage degenerations such as meniscus, osteoporosis.

9. The use according to claim 8, whereby the preparation used for joint and tendons calcification also comprises specific calcium dissolving minerals, including, without being limited to, acetic acid, citric acid, EDTA (ethylenediaminetetracetic acid), sodium bicarbonate and sodium hydroxide.

10. The use of a preparation according to claim 1, applied transdermally via transporation to a subject for preventing, controlling, mitigating or treating metabolic diseases such as lymphatic (diabetes, ageing) and circulatory problems (varicose veins, heavy legs, etc.), obesity, fat accumulation and metabolism.

11. The use of a preparation according to claim 1, applied transdermally via transporation to a subject for preventing, controlling, mitigating or treating organ-centered degenerations, including, without being limited to, hepatitis, gallbladder and kidney stones, intestinal and respiratory problems.

12. The use of a preparation according to claim 1, wherein the quantity of preparation applied transdermally is comprised from 0.1 to 500 gr and preferably of approximately 1 to 10 grs., according to the size of the area to be treated.

13. The use of a preparation according to claim 1, wherein the composition is in the form of a powder to be dissolved in distilled water, or in the form of a creme, gel or liquid suitable for administration by transporation methods.

14. The use according to claim 1, wherein the subject is an animal or human subject.
